(19) United States
(12) Patent Application Publication
(10) Pub. No.: US 2019/0015082 A1
(43) Pub. Date: Jan. 17, 2019

(54) Urine Collection Holder

(71) Applicant: Michelle P. Little, Farmers Branch, TX (US)

(72) Inventor: Michelle P. Little, Farmers Branch, TX (US)

(21) Appl. No.: 15/650,870

(22) Filed: Jul. 15, 2017

(51) Int. Cl. A61B 10/00; A61B 90/50

(52) U.S. Cl. CPC A61B 10/007; A61B 90/50

(57) ABSTRACT

Disclosed is a disposable device for collecting a urine specimen. The device comprises a collection cup and an elongate, flat, foldable holder, which is further divided into a head comprising an opening for snugly receiving the cup therewithin and an elongate, foldable handle. The handle further comprises a grip section and a middle section connecting the grip section and the head. The holder is adapted to be foldably configurable such that, the inclination between the grip section and the middle section and the head and the middle section is lockable at a proximal angle and a distal angle respectively. The cup, upon being received within the opening, is as enabled by handling of the device about the grip section, positioned underneath the urethra of a patient to collect a sterile specimen of the urine emanating therefrom.

### BACKGROUND

### Background of the Invention

### 1. Field of the Invention

The present invention generally relates to an apparatus for collecting urine sample. More specifically, the invention relates a foldable holder for hygienically holding a container used for collecting urine sample without soiling user's hands or body.

### 2. Description of Related Art

Urine sample is commonly used in clinical diagnosis of disease conditions and physiological changes. In order to obtain urine sample, a patient or a caretaker of the patient has to position and hold the sample container using bare hands. Currently, it is difficult, especially for female patients to capture urine sample in a small bottle or sample collection cup. One of the major problems involves proper positioning of the container for collecting adequate amount of urine sample. It is difficult especially for nurse or caretaker to properly position the container and collect urine sample from a patient without spattering of urine on hands, which is unhygienic and discomforting. Another problem is related to contamination of urine sample with foreign objects when bare hands are used to hold sample container.

Generally, while collecting urine sample, it is easy to miss the mouth of the sample container, which might lead to spattering of urine on the body surface. In some cases, insufficient collection of sample into the container might require an additional visit to the laboratory or testing clinic. There exists numerous devices, which are used for collecting urine sample from patients. Many of these devices involve funnel like configurations that are positioned to catch a stream of urine and direct it to a collection container. Multi-chambered apparatus are also used for collecting urine samples. However, these devices generally utilize plastic molded materials that are reused for different patients, which might lead to spreading of infections.

Therefore, there is a need for an improved urine sample collecting apparatus, which is disposable, and made of low cost material. There is also a need for a sample collection apparatus, which allows easy and hygienic collection of urine sample.

### SUMMARY

An embodiment of the present invention comprises a disposable device for collecting a sterile urine specimen for clinical diagnosis. More particularly, the device is intended for easily and accurately positioning itself underneath a urethra and thereafter for cleanly collecting said urine specimen without said specimen spattering on one's hands. The device comprises a cylindrical urine specimen collection cup and an elongate, flat, foldable holder wherein, a configured folder and the cup are assembled together to form the device. The folder comprises a unitary piece broadly divided into a foldable head and an elongate, foldable handle.

The head comprises a substantially rectangular central head portion flanked by a pair of elongate, rectangular guide flaps. The central head portion comprises an opening centrally disposed thereon. The opening, which is circular in shape, comprises a plurality of tapered projections extending inwardly (towards the center of the opening) from the circumference thereof. The opening is adapted to receive the cup therewithin and the projections help achieve a snug fit therebetween. The guide flap is, with respect to the central head portion, adapted to be foldable about a crease line. A first retainer tab extends from the longitudinal proximal extremity of each guide flap. The first retainer tab is foldable about the corresponding guide flap. The free extremity of the first retainer tab comprises a locking extremity.

The handle is further divided into an elongate grip section and an elongate, rectangular middle section wherein, the middle section connects the head and the grip section. The grip section is in turn divided into an elongate, rectangular central grip portion and a pair of opposingly-disposed, elongate second retainer tabs flanking either side of the central grip portion. The second retainer tab is foldable with respect to the central grip portion about a crease line. The longitudinal distal extremity of the second retainer tab comprises a locking extremity. The middle section comprises two pairs of opposingly-disposed first and second tab slots closer to the longitudinal edges thereof. The first and second pairs of slots are adapted to lockingly receive the locking extremities of the first and second retainer tabs respectively.

In order to foldably configure the flat holder, the middle section is folded upwards to an approximate angle of 45° with respect to the head. Notably, this angle of 45° is referred to as the distal angle hereinafter. Once there, the guide flaps are bent upwards to an approximate angle of 30°. Thereafter, the first retainer tabs are bent inwardly sideward until the locking extremities of the first retainer tabs are lockingly received within the first tab slots. Once the first retainer tabs are locked in place, the distal angle is retained. The middle section is folded downwards to an approximate angle of 45° with respect to the grip section. Notably, this angle of 45° is referred to as the proximal angle hereinafter. Once there, the second retainer tabs are folded downwards to a 90°-angle and the locking extremities of the second retainer tabs are lockingly received within the second tab slots. Once the second retainer tabs are locked in place, the proximal angle is retained. Once the folder is thus foldably configured, the head and the grip section are substantially parallel to each other, while the middle section is rendered oblique.

In order to collect the urine specimen, the device is held about the grip section and the head (assembled with the cup), about the guide flaps, is effectively guided between the upper portion of thighs until the cup is aligned with the urethra. Once aligned, sterile urine specimen is deposited within the cup whereafter, the cup is sent for clinical analysis.

Other features and advantages will become apparent from the following description of the preferred embodiments, taken in conjunction with the accompanying drawings.

### BRIEF DESCRIPTION OF THE DRAWINGS

Various embodiments of the disclosed system and method are described herein with reference to the accompanying drawings, which form a part of this disclosure, wherein:

FIG. 1, according to an embodiment of the present invention, is an illustration of a plan view of the holder.

FIG. 2, according to an embodiment of the present invention, is an illustration of a plan view of the head.

FIG. 3, according to an embodiment of the present invention, is an illustration of a plan view of the central head portion.

FIG. 4, according to an embodiment of the present invention, is an illustration of a plan view of the grip section.

FIG. 5, according to an embodiment of the present invention, is an illustration of a plan view of the middle section.

FIG. 6, according to an embodiment of the present invention, is an illustration of a perspective view of the configured holder.

FIG. 7, according to an embodiment of the present invention, is an illustration of a side view of the configured holder.

FIG. 8, according to an embodiment of the present invention, is an illustration of a perspective view of the collection device.

FIG. 9, according to an embodiment of the present invention, is an illustration of a side view of the collection device.

FIG. 10, according to an embodiment of the present invention, is an illustration of a seated user employing the device.

FIG. 11, according to an embodiment of the present invention, is an illustration of a standing user employing the device.

FIGS. 12A through 12C, according to an embodiment of the present invention, are sequential illustrations of a holder being taken out from the package box.

The various aspects of the present disclosure mentioned above are described in further detail with reference to the aforementioned figures and the following detailed description of exemplary embodiments.

### FIGURES—REFERENCE NUMERALS

10—Disposable Urine Specimen Collection Device
12—Foldable Holder
14—Urine Specimen Collection Cup
16—Head
18—Gripping Section
20—Middle Section
22—Proximal Crease Line
24—Distal Crease Line
26—Central Head Portion
28—Guide Flap
30—Side Crease Line
32—First Retainer Tab
34—Tab Crease Line
36—Opening
38—Projection
39—Central Grip Portion
40—Second Retainer Tab
42—Tab Slot
44—Package Box
46—Dispense Tab

### DETAILED DESCRIPTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

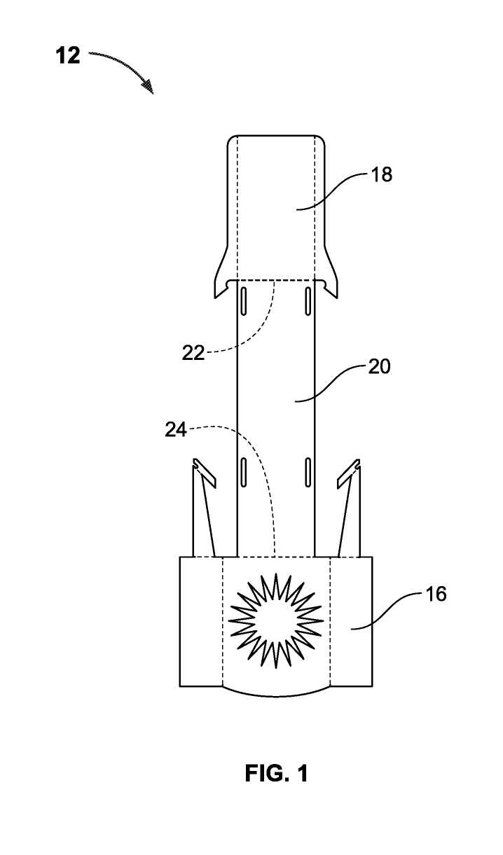

Embodiments of the present invention are directed to a disposable device for collecting a urine specimen for clinical diagnosis. More particularly, the device is intended for easily and accurately positioning itself underneath a urethra and thereafter for cleanly collecting said urine specimen without spattering on one's hands. Referring to FIGS. 1, 8 and 9, the device 10, which is simple in construction, comprises a cylindrical urine specimen collection cup 14 and an elongate, flat, foldable holder 12. The folder 12 and the cup 14 are intended to be assembled together to form the device 10. Notably, the holder 12 is preferably made of a biodegradable heavy-duty paper, such as a cardboard material. Preferably, the holder 12 is made of cardboard sheet of 1/16, which preferably is corrugated. The folder 12 is formed using a specially designed steel rule die and a paper press. The folder 12, which is a unitary piece, is broadly divided into a foldable head 16 and an elongate, foldable handle (not labeled).

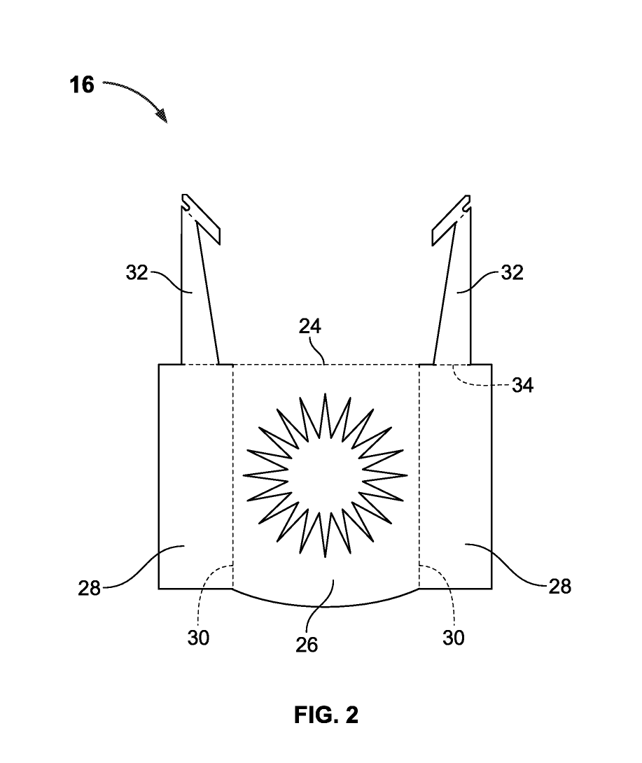

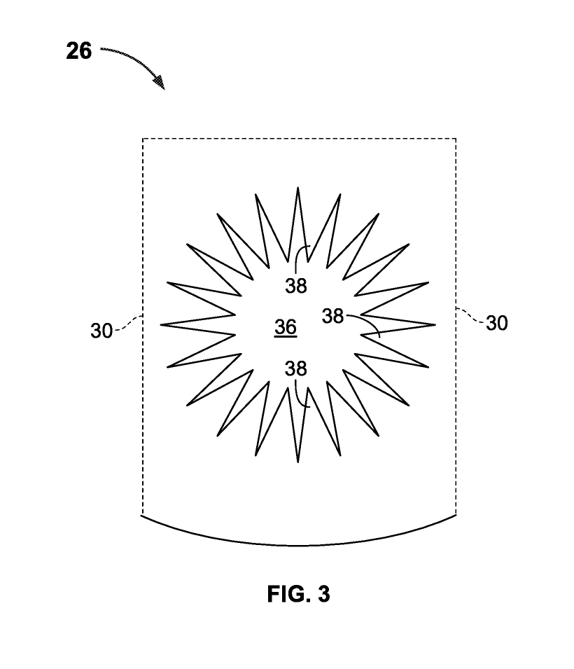

Referring to FIGS. 1 through 3, the head 16 comprises a substantially rectangular central head portion 26 flanked by a pair of elongate, rectangular guide flaps 28. As can be appreciated from FIG. 3, the central head portion 26 comprises an opening 36 centrally disposed thereon. The opening 36, which is circular in shape, comprises a plurality of tapered projections 38 extending inwardly (towards the center of the opening 36) from the circumference thereof. Notably, each projection 38 terminates in a pointed extremity. The opening 36 is adapted to receive the cup 14 therewithin and the projections 38 help achieve a snug fit between the cup 14 and the opening 36. In one embodiment, the cup 14 comprises a circular lip (not shown) protruding outwardly from the brim thereof wherein, the lip prevents the cup 14 from falling through the opening 36. Notably, the opening 36 is intended to hold any standard urine collection cup employed in the art as the projections 38 ensure a snug fit thereof when received within the opening 36.

Still referring to FIGS. 1 through 3, a side crease line 30 separates each guide flap 28 from the central head portion 26. The guide flap 28 is, with respect to the central head portion 26, adapted to be foldable about the side crease line 30 wherein, the utility of said folding will become apparent from the following body of text. A first retainer tab 32 extends from the longitudinal proximal extremity of each guide flap 28. The first retainer tab 32 and the corresponding guide flap 28 are separated by tab crease line 34 rendering the first retainer tab 32 foldable about the corresponding guide flap 28. The first retainer tab 32 comprises an elongate tapered member, the free extremity of which comprises a locking extremity, the utility of which will become apparent from the following body of text.

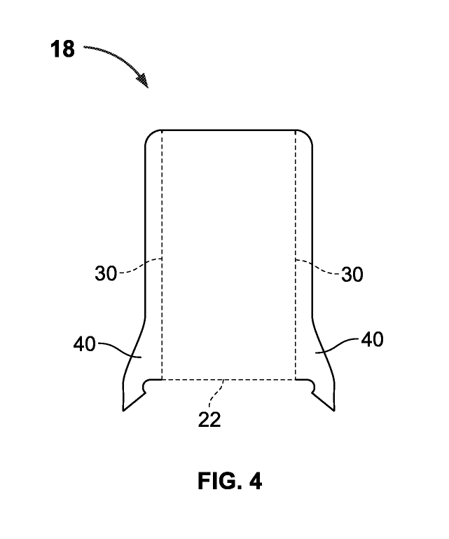

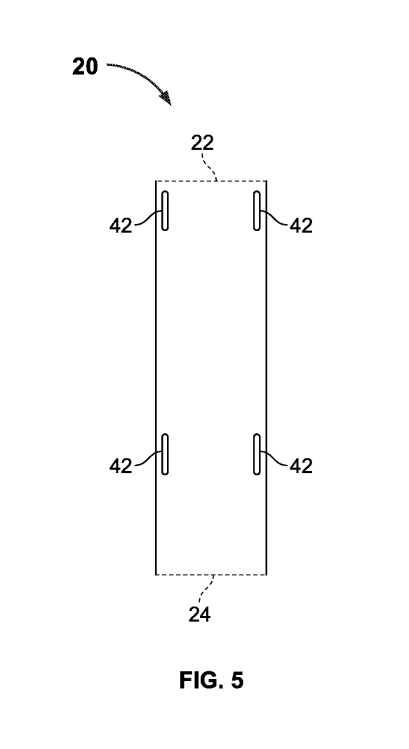

Referring to FIGS. 1, 4 and 5, the handle is further divided into an elongate grip section 18 and an elongate, rectangular middle section 20 wherein, the middle section 20 connects the head 16 and the grip section 18. The grip section 18 is in turn divided into an elongate, rectangular central grip portion 39 and a pair of opposingly-disposed, elongate second retainer tabs 40 flanking either side of the central grip portion 39. A side crease line 30 separates each second retainer tab 40 from the central grip portion 39 whereby, the second retainer tab 40 is foldable with respect to the central grip portion 39 about the side crease line 30. The longitudinal distal extremity of the second retainer tab 40 comprises a locking extremity, the utility of which will become apparent from the following body of text. Notably, the width of the longitudinal distal extremity second retainer tab 40 is wider than the rest thereof. The middle section 20, the width of which is equivalent to that of the central grip portion 39, comprises two pairs of opposingly-disposed first and second tab slots 42 closer to the longitudinal edges thereof. The first and second pairs of slots 42 are adapted to lockingly receive the locking extremities of the first and second retainer tabs 32 and 40 respectively.

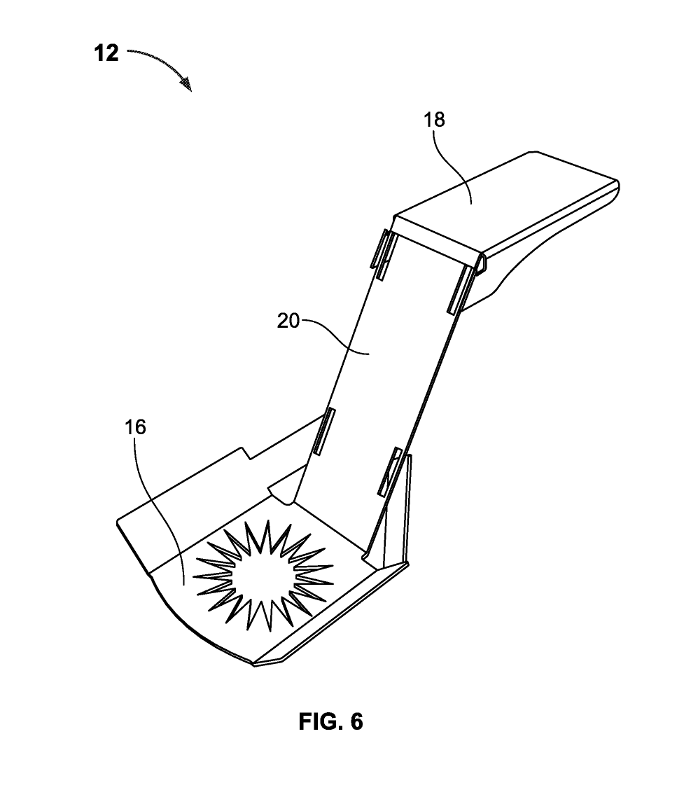

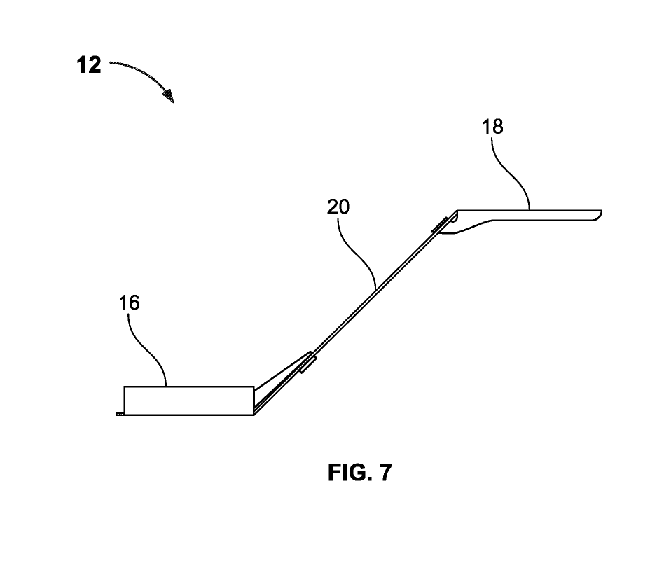

Referring to FIGS. 6 and 7, in order to foldably configure the flat holder 12, the middle section 20, about the distal crease line 24, is folded upwards to an approximate angle of 45° with respect to the head 16. Notably, this angle of 45° is referred to as the distal angle hereinafter. Once there, the guide flaps 28, about the side crease lines 30, are bent upwards to an approximate angle of 30°. Thereafter, the first retainer tabs 32, about the tab crease lines 34, are bent inwardly sidewards until the locking extremities of the first retainer tabs 32 are lockingly received within the first tab slots 42. More particularly, the locking extremities are received within the first tab slots 42 from the backside or underside of the middle section 20. Once the first retainer tabs 32 are locked in place, the distal angle between the head 16 and the middle section 20 is retained.

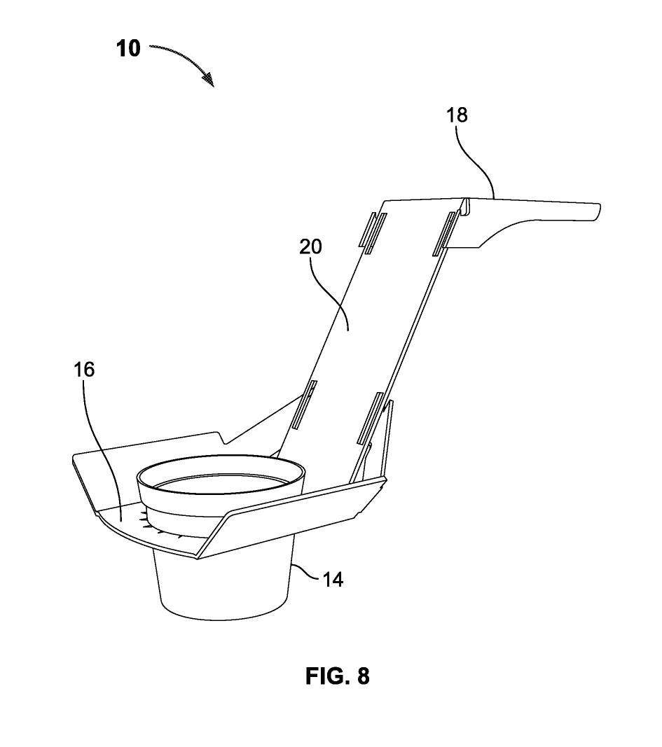

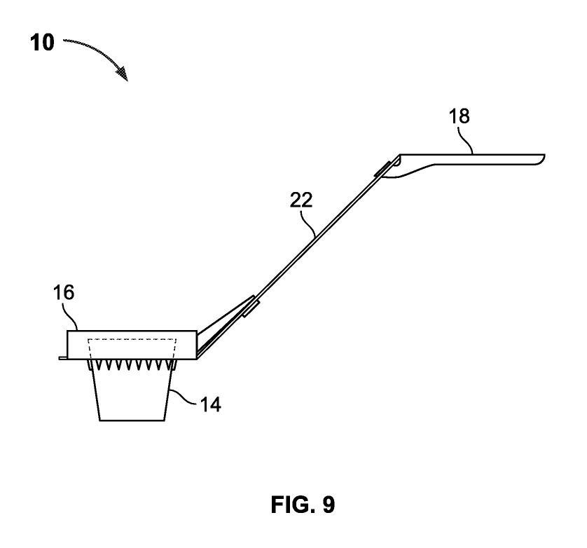

Still referring to FIGS. 6 and 7, the middle section 20, which is now one with the head 16 by virtue of being locked therewith, is, about the proximal crease line 22, folded downwards to an approximate angle of 45° with respect to the grip section 18. Notably, this angle of 45° is referred to as the proximal angle hereinafter. Once there, the second retainer tabs 40 are folded downwards to a 90°-angle and the locking extremities of the second retainer tabs 40 are lockingly received within the second tab slots 42 from the backside or underside of the middle section 20. Once the second retainer tabs 40 are locked in place, the proximal angle between the grip section 18 and the middle section 20 is retained. Once the folder 12 is thus foldably configured, the head 16 and the grip section 18 are substantially parallel to each other, while the middle section 20 obliquely disposed at an angle of 45°. As can be appreciated from FIGS. 8 and 9, the device 10 is formed when the cup 14 is assembled with the holder 12.

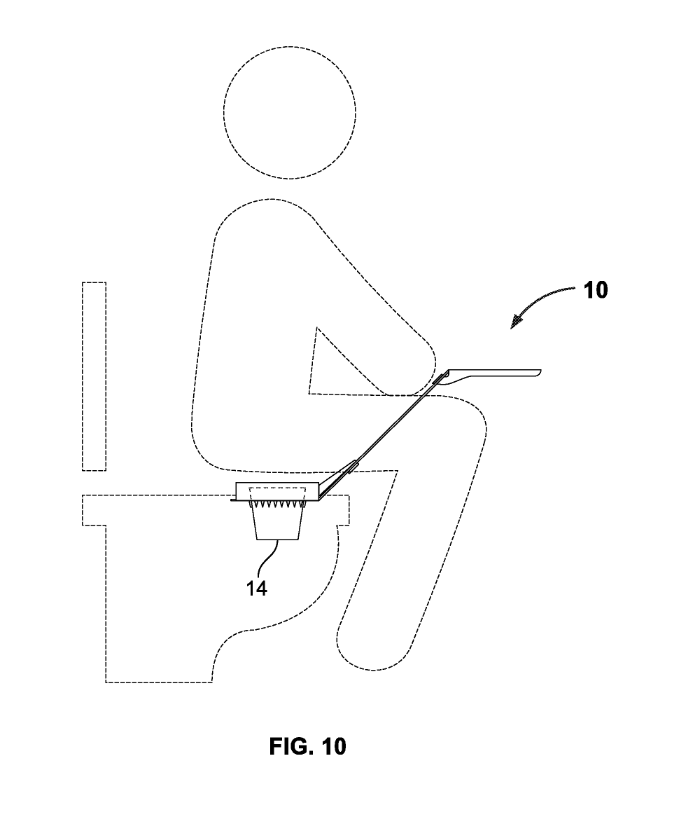

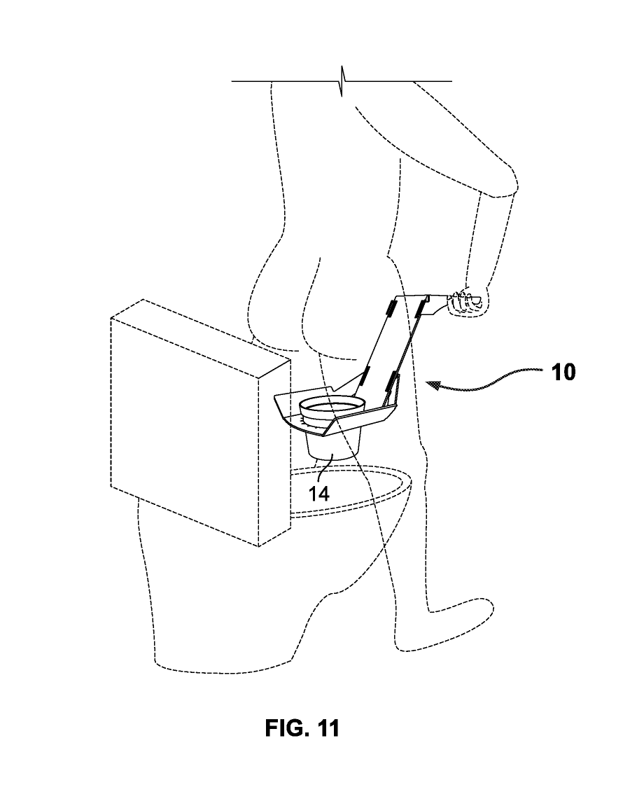

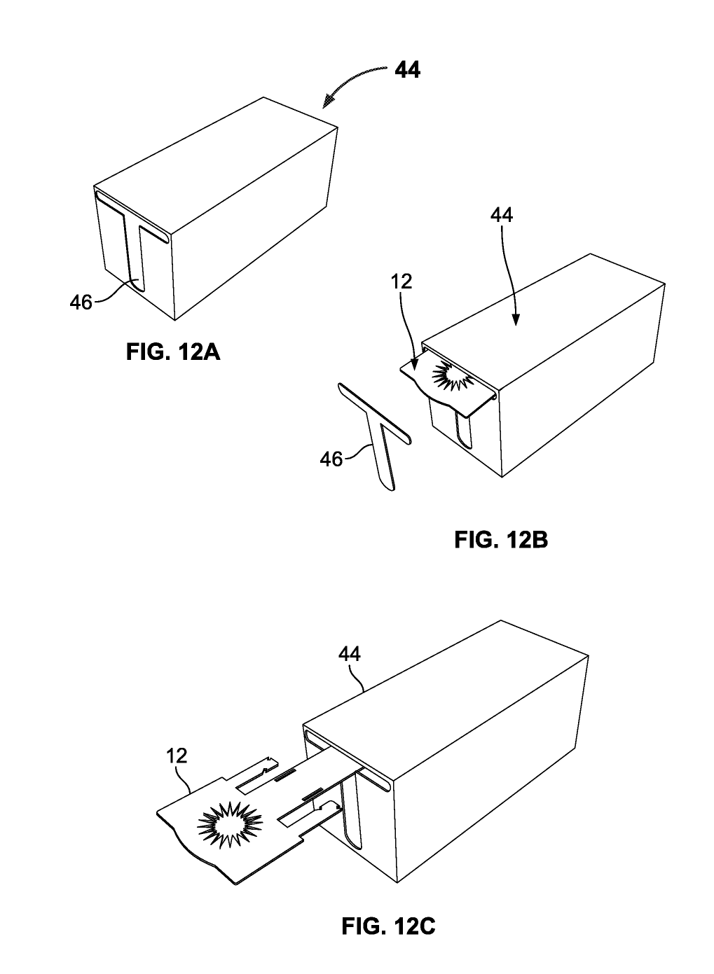

Referring to FIGS. 10 and 11, in order to collect the urine specimen, the device 10 is held about the grip section and the head (assembled with the cup 14), about the guide flaps, is effectively guided between the upper portion of thighs until the cup is aligned with the urethra. This is irrespective of the user being in a seated or a standing position. Once aligned, sterile urine specimen is deposited within the cup 14 whereafter, a lid is placed over the cup 14 and the cup 14 is sent for clinical analysis. Notably, during the collection of the specimen, the guide flaps shield the user's skin against any inadvertent splashing and sputtering. After use, the device 10 can be easily unfolded and recycled to reduce the impact on the environment. As can be appreciated from FIGS. 12A through 12C, the unassembled device 10 is packaged in a rectangular package box 44. The package box 44 comprises a T-shaped opening disposed on longitudinal side thereof wherein, the T-shaped opening is covered by a removable T-shaped dispense tab 46. A plurality of folders 12 are stacked over one another within the package box 44 wherein, upon the removal of the T-shaped tab 46, the folders 12 are slid out one after another to employ the same.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

1. A disposable urine specimen collection device comprising:
(a) a urine specimen collection cup; and
(b) an elongate, flat, foldable holder comprising:
(i) a head comprising an opening for snugly receiving the cup therewithin by downwardly extending projections; and
(ii) an elongate, foldable handle comprising:
(1) a grip section; and
(2) a middle section connecting the grip section and the head;
wherein, the holder is adapted to be foldably configurable such that, the inclination between the grip section and the middle section and the head and the middle section is lockable at a proximal angle and a distal angle respectively; the cup, upon being received within the opening, as enabled by handling of the device about the grip section, positioned underneath the urethra of a patient to collect a sterile specimen of the urine that emanates therefrom; and
wherein the head includes a central head portion including an opening centrally disposed thereon; and
a pair of opposingly-disposed guide flaps flanking either side of the central head portion.

2. (canceled)

3. The device of claim 1 wherein, the holder comprises a unitary piece.

4. The device of claim 1 wherein, the handle is folded about crease lines preformed thereon.

5. The device of claim 1 wherein, upon foldably configuring the holder, the head and the grip section are substantially parallel to one another, while middle section is rendered oblique; the head being in a plane lower than that of the grip section.

6. The device of claim 1 wherein, the plurality of tapered projections extending inwardly from a circumference thereof, the projections to bend downwardly upon receiving the cup within the opening so as to achieve a snug fit between the cup and the opening.

7. The device of claim 1 wherein, the head comprises:
(a) a first retainer tab extending from a longitudinal proximal extremity of each guide flap, a proximal extremity of the first retainer tab adapted to be removably locked into the middle section resulting in the corresponding guide flap being bent upwards obliquely with respect to the middle section and the head being inclined at the distal angle with respect to the middle section.

8. The device of claim 7 wherein, the middle section comprises a first pair of tab slots for lockingly receiving the proximal extremities of the first retainer tabs.

9. The device of claim 1 wherein, the grip section comprises:
(a) a central grip portion; and
(b) a pair of opposingly-disposed second retainer tabs flanking either side of the central grip portion, a distal extremity of each second retainer tab adapted to be removably locked into the middle section resulting in the grip section being inclined at the proximal angle with respect to the middle section.

10. The device of claim 9 wherein, the middle section comprises a second pair of tab slots for lockingly receiving the distal extremities of the second retainer tabs.

11. The device of claim 1 made of heavy-duty paper.

12. The device of claim 11 made of corrugated cardboard material.

13. The device of claim 1 made of a material that is biodegradable.

14. A disposable urine specimen collection device comprising:
(a) a urine specimen collection cup; and
(b) an elongate, flat, foldable holder comprising a unitary piece, the holder comprising:
(i) a head comprising an opening for snugly receiving the cup therewithin by downwardly extending projections; and
(ii) an elongate, foldable handle comprising:
(1) a grip section; and
(2) a middle section connecting the grip section and the head;
wherein, the holder is adapted to be foldably configurable about crease lines such that, the inclination between the grip section and the middle section and the head and the middle section is lockable at a proximal angle and a distal angle respectively, upon foldably configuring the holder, the head and the grip section are substantially parallel to one another, while middle section is rendered oblique with respect to the head, the head being in a plane lower than that of the grip section; the cup, upon being received within the opening, as enabled by handling of the device about the grip section, positioned underneath the urethra of a patient to collect a sterile specimen of the urine that emanates therefrom;
wherein the head includes a central head portion including
an opening centrally disposed thereon; and
a pair of opposingly-disposed guide flaps flanking either side of the head.
